(19) United States
(12) Patent Application Publication
(10) Pub. No.: US 2002/0037884 A1
(43) Pub. Date: Mar. 28, 2002

(54) Topical composition comprising ciprofloxacin and hydrocortisone

(75) Inventors: Onkar N. Singh, Arlington, TX (US); Haresh G. Bhagat, Fort Worth, TX (US); Bhagwati P. Kabra, Arlington, TX (US)

Correspondence Address: Alcon Universal Ltd., c/o Alcon Research, Ltd, Patrick M. Ryan(Q-148), R&D Counsel, 6201 So. Freeway, Fort Worth, TX, 76134-2099, US

(73) Assignee: Alcon Universal Ltd.

(21) Appl. No.: 09/906,325

(22) Filed: Jul. 16, 2001

Related U.S. Application Data
(60) Provisional application No. 60/220,929, filed on Jul. 26, 2000.

(51) Int. Cl. A61K 31/573; A61K 31/545

(52) U.S. Cl. 514/171; 514/200

(57) ABSTRACT

This invention is directed toward a topical composition comprising ciprofloxacin and hydrocortisone, where the composition contains a specific grade of polyvinyl alcohol as a viscosity augmenter. The specified grade of polyvinyl alcohol is 85-90% hydrolyzed polyvinyl alcohol.

This application claims priority to U.S. Provisional Application, Ser. No. 60,220,929, filed Jul. 26, 2001.

### BACKGROUND OF THE INVENTION

The present invention relates to topically administrable ophthalmic and otic pharmaceutical compositions. In particular, this invention relates to compositions comprising ciprofloxacin and hydrocortisone in combination with 85-90% hydrolyzed polyvinyl alcohol as a viscosity augmenter.

### DESCRIPTION OF RELATED ART

U.S. Pat. No. 5,843,930 discloses topically administrable ophthalmic and otic compositions comprising (a) ciprofloxacin in aqueous solution in an amount effective for antibacterial action; (b) a non-ionic viscosity augmenter unaffected by pH and ionic level, said viscosity augmenter being present in an amount effective for augmenting the viscosity of the composition to a viscosity greater than that of water, said viscosity augmenter being at least 85% hydrolyzed polyvinyl alcohol; (c) a non-ototoxic preservative present in an amount effective for antibacterial action the preservative being benzyl alcohol; (d) water sufficient to produce an aqueous composition; (e) hydrocortisone in aqueous suspension in an amount effective for anti-inflammatory action; (f) lecithin in an amount effective for enhancing suspension of other constituents in the compositions; and (g) polysorbate ranging from polysorbate 20 to 80 in an amount effective for spreading the preparation on a hydrophobic skin surface to the site of infection or inflammation.

According to the '930 patent, the compositions comprising ciprofloxacin and hydrocortisone contain polyvinyl alcohol in an amount effective for augmenting the viscosity of the composition to a viscosity greater than that of water and suspending other constituents of the composition. To allow a ciprofloxacin preparation to be administered in drops from a medicine dropper and to flow by gravity to and remain or deposit in an effective amount at a selected area, a viscosity-augmenting agent which would also serve to suspend hydrocortisone was desirable. For compatibility with ciprofloxacin hydrochloride solubility, viscosity-augmenting agents were preferably non-ionic and unaffected by pH and ionic level. See Col., 8, lines 13-31 of the '930 patent.

Polyvinyl alcohol was selected for its ability to produce a suitable viscosity and a high ability to suspend hydrocortisone in aqueous preparations. See the '930 patent at Col. 8, lines 32-37.

Concerning the selection of specific grades of polyvinyl alcohol, the '930 patent discloses at Col. 8, lines 46-63:

In comparisons with compositions with fully dissolved polyvinyl alcohol, compositions with partially dissolved polyvinyl alcohol showed fewer strokes and larger sedimentation volume. However, because of anticipated variability and change in the amount dissolved over varying temperature conditions expected to occur in storage, compositions with fully dissolved polyvinyl alcohol were preferred. Polyvinyl alcohol in an 85% hydrolyzed grade was effective in suspending hydrocortisone. However, polyvinyl alcohol in a medium viscosity grade, 99% hydrolyzed, was determined to be superior in suspending hydrocortisone. Such material is commercially available under the tradename Airvol 125 from Air Products and Chemicals Inc. Amount of polyvinyl alcohol effective to augment the viscosity of and to suspend hydrocortisone in aqueous compositions with ciprofloxacin hydrochloride range from about 0.1 to about 10 weight percent, preferably from about 1 to about 5 weight percent, and most preferably about 2 weight percent.

### SUMMARY OF THE INVENTION

The present invention is directed toward topical compositions for the treatment of otitis externa and otitis media. The compositions are suspension compositions comprising polyvinyl alcohol of a grade that is 85-90% hydrolyzed. The present compositions posses superior physical stability relative to similar compositions comprising polyvinyl alcohol of a grade that is greater than 90% hydrolyzed, such as the preferred 99% hydrolyzed polyvinyl alcohol disclosed in the '930 patent.

Among other factors, the present invention is based on the finding that ciprofloxacin and hydrocortisone suspension compositions containing polyvinyl alcohol of a grade that is 85-90% hydrolyzed are significantly less likely to leave polymeric deposits or residue after drying.

### BRIEF DESCRIPTION OF THE DRAWINGS

FIG. 1 compares two ciprofloxacin/hydrocortisone formulations containing different grades of polyvinyl alcohol (0.05% each) after standing for 2 hours.

FIG. 2 compares two ciprofloxacin/hydrocortisone formulations containing different grades of polyvinyl alcohol (2.0% each) after standing for 45 minutes.

FIG. 3 shows the physical stability of a ciprofloxacin/hydrocortisone formulation containing 0.05% of an 88% hydrolyzed grade of polyvinyl alcohol (Airvol 205S) over time.

FIG. 4 shows the physical stability of a ciprofloxacin/hydrocortisone formulation containing 0.05% of a 99% hydrolyzed grade of polyvinyl alcohol (Airvol 125) over time.

FIG. 5 shows ciprofloxacin/hydrocortisone formulations with varying concentrations of an 88% hydrolyzed grade of polyvinyl alcohol (Airvol 205S) after standing for 1 hour.

FIG. 6 shows ciprofloxacin/hydrocortisone formulations with varying concentrations of a 99% hydrolyzed grade of polyvinyl alcohol (Airvol 125) after standing for 1 hour.

### DESCRIPTION OF THE INVENTION

Unless otherwise indicated, all ingredient concentrations are listed as percent (w/w).

Ciprofloxacin is present in the compositions of the invention in an amount effective for anti-bacterial action. Such amounts range from about 0.01 to about 1%, preferably from about 0.1 to about 0.5%, and most preferably about 0.2%. Compositions of the present invention also comprise hydrocortisone as an anti-inflammatory agent. Hydrocortisone is present in an amount effective for anti-inflammatory action. Such amount typically ranges from about 0.1 to about 3%, preferably about 0.1 to about 2%, and most preferably about 1%. Particularly for ophthalmic use, small particle sizes are preferred. As used herein, “micronized” hydrocortisone means hydrocortisone particles having an average particle size≦10 (μm (based on surface area (dsn)). If the particle size of the hydrocortisone raw material as received from the supplier is unsatisfactory, one or more known sizing techniques, such as ball milling or micronizing, can be used to adjust the particle size into the desired range.

To prevent contamination by microorganisms and provide a reasonable shelf-life, the compositions of the present invention include a preservative. Acceptable preservatives are required to cause no or insignificant ototoxicity, sensitization or irritation of the ear. Additionally, the preservative must be jointly soluble with ciprofloxacin in water over a pH range of approximately pH 3-6. The most preferred preservative is benzyl alcohol, which is typically present in an amount from about 0.1 to 3%, preferably about 0.1 to 2%, and most preferably about 0.9%.

A tonicity adjusting agent is preferably contained in an amount sufficient to cause the composition to be approximately isotonic, that is an amount effective to adjust the tonicity of the composition from about 150 to about 800 mOsm, preferably 200-600 mOsm. A preferred tonicity-adjusting agent is sodium chloride.

A buffering agent is desirable for the compositions of the present invention. The preferred buffering system is an acetate buffer comprising acetic acid and sodium acetate. Amounts of sodium acetate and acetic acid effective to buffer the preparation in a pH range of about 4.0-5.3, preferably about 4.4-4.9 and most preferably about 4.7, range from about 0.1 to about 3% of sodium acetate and from about 0.01 to about 10% of acetic acid. Preferably the amount of sodium acetate is from about 0.1 to about 2% and most preferably about 0.4%. Preferably the amount of sodium acid is about 0.1 to 5% and most preferably about 0.7%. Sodium acetate is preferably used in the form of sodium acetate trihydrate and acetic acid is preferably used in the form of glacial acetic acid.

To allow the compositions of the present invention to wet and spread on the skin surface at the site of infection or inflammation in the ear canal, a non-ionic surfactant is desirable. The surfactants known as polysorbates, in particular polysorbates 20-80, are preferred. Such polysorbates are commercially available under the tradename Tween from ICI Americas, Inc. Most preferred is polysorbate 20. The amount of polysorbate surfactant contained in the compositions of the present invention generally ranges from about 0.01 to about 2%, preferably about 0.05 to about 1%, and most preferably about 0.1%.

To help maintain or improve the physical stability of the suspension composition of the present invention, lecithin is added. Lecithin is commercially available in at least two grades: a fully hydrogenated soy lecithin comprising 90% phosphatidylcholine, which is commercially available under the tradename Phospholipon 90H from American Lecithin Company, and a soy lecithin comprising 75% phosphatidylcholine, which is commercially available under the tradename Lipoid-S75 from Vernon Walden, Inc. In general, the amount of lecithin contained in the compositions of the present invention will range from about 0.01 to about 5%, preferably about 0.01 to about 2% and most preferably about 0.15%.

In addition to the excipients mentioned above, the ciprofloxacin and hydrocortisone compositions of the present invention comprise polyvinyl alcohol as a viscosity-augmenting agent. The polyvinyl alcohol contained in the composition of the present invention is an 85-90% hydrolyzed grade. Preferably, the polyvinyl alcohol ingredient is an 88% hydrolyzed grade, such as that commercially available as Airvol 205S from Air Products and Chemicals, Inc. The amount of polyvinyl alcohol ingredient contained in the compositions of the present invention is an amount effective to cause the composition to have a viscosity ranging from about 2 to about 8 cps (when measured at room temperature using a Brookfield Viscometer set at 30 rpm and a CP 42 spindle). Preferably, the polyvinyl alcohol ingredient is present in an amount sufficient to cause the composition's viscosity to be from about 3 to 7 cps.

According to a preferred method for preparing the suspension compositions of the present invention, lecithin and surfactant (50% of the required amounts of each) are dispersed in purified water at a temperature of about 65-70° C., then removed from heat. Micronized hydrocortisone is added while the lecithin dispersion is cooling and the resulting mixture is mixed overnight (approximately 12 hours). Separately, a ciprofloxacin solution is prepared by adding the following components in order, allowing each to fully disperse or dissolve before the next is added: remaining 50% of lecithin, remaining 50% of surfactant, preservative, buffer (e.g., glacial acetic acid then sodium acetate (trihydrate)), ciprofloxacin, and the tonicity-adjusting agent. The hydrocortisone dispersion is added to the ciprofloxacin solution, then the required amount of polyvinyl alcohol is added from a polyvinyl alcohol stock solution.

The following examples are presented to illustrate further various aspects of the present invention, but are not intended to limit the scope of the invention in any respect.

### EXAMPLES

### Examples 1-11

Each of the formulations in Table 1 below was prepared as follows. For a 200 ml batch size, formulations 1-6 were prepared using Method A and formulations 7-11 were prepared using Method B.

Method A

1. Tare an appropriate sized compounding bottle (e.g., 250 mL) with stir bar.

2. Add the correct amount of polyvinyl alcohol (PVA) to the compounding bottle from a 5% stock solution.

3. Add 50% of the total batch volume of purified water to the compounding bottle and heat to 90° C. while stirring.

4. Add the correct amount of lecithin (phospholipon 90H) to the heated PVA solution and disperse by stirring.

5. Allow the lecithin dispersion to cool to room temperature.

6. Add the remaining ingredients in the following order, allowing each to disperse/dissolve by stirring before adding the next: benzyl alcohol, glacial acetic acid, sodium acetate, polysorbate 20, ciprofloxacin hydrochloride, hydrocortisone and sodium chloride.

7. Measure and adjust pH to target pH 4.7±0.2 with 1N NaOH or 1N HCl, if necessary.

8. QS to 100% total batch volume with purified water.

Method B

Part I

1. Tare an appropriate sized compounding bottle (e.g., 250 mL) with stir bar.

2. Add the correct amount of polyvinyl alcohol (PVA) to the compounding bottle from the 5% stock solution.

3. Add 50% of the total batch volume of purified water to the compounding bottle and heat to 90° C. while stirring.

4. Add the correct amount of lecithin (pholspholipon 90H) to the heated PVA solution and disperse by stirring.

5. Allow the lecithin dispersion to cool to room temperature.

Part II

1. In a 100 mL beaker weigh the required amount of polysorbate 20. Add a small amount of purified water to the beaker and begin to stir.

2. Weigh and add the required amount of hydrocortisone (micronized) to the beaker.

3. Allow the hydrocortisone to mix well with the polysorbate 20 solution.

Part III

1. Once the mixture in Part I has cooled to room temperature, add the ingredients in the following order, allowing each to disperse/dissolve before adding the next: benzyl alcohol, glacial acetic acid, sodium acetate, ciprofloxacin hydrochloride and sodium chloride.

2. Add the hydrocortisone slurry from Part II to Part I.

3. Mix well by stirring.

4. Measure and adjust pH to 4.7±0.2 with 1N NaOH or 1 N HCl, if necessary.

5. QS to 100% with purified water.

TABLE 1A

| | EXAMPLE | | | | | |
|---|---|---|---|---|---|---|
| INGREDIENT | 1 | 2 | 3 | 4 | 5 | 6 |
| Ciprofloxacin | 0.35* | 0.35* | 0.35* | 0.35* | 0.35* | 0.35* |
| Hydrochloride, | | | | | | |
| monohydreate | | | | | | |
| Hydrocortisone, | 1.0 | 1.0 | 1.0 | 1.0 | 1.0 | 1.0 |
| micronized | | | | | | |
| Benzyl alcohol | 0.9 | 0.9 | 0.9 | 0.9 | 0.9 | 0.9 |
| Polyvinyl Alcohol | — | 0.05 | 0.1 | 0.5 | 1.0 | 2.0 |
| (AIRVOL 125) 99% | | | | | | |
| hydrolyzed | | | | | | |
| Sodium Chloride | 0.9 | 0.9 | 0.9 | 0.9 | 0.9 | 0.9 |
| Sodium Acetate, Trihydrate | 0.68 | 0.68 | 0.68 | 0.68 | 0.68 | 0.68 |
| Glacial Acetic acid | 0.255 | 0.255 | 0.255 | 0.255 | 0.255 | 0.255 |
| Lecithin (Phospholipon 90H) | 0.15 | 0.15 | 0.15 | 0.15 | 0.15 | 0.15 |
| Polysorbate 20 | 0.10 | 0.10 | 0.10 | 0.10 | 0.10 | 0.10 |
| Sodium Hydroxide | pH adjust | pH adjust | pH adjust | pH adjust | pH adjust | pH adjust |
| and/or Hydrochloric acid | to 4.7 | to 4.7 | to 4.7 | to 4.7 | to 4.7 | to 4.7 |
| Purified Water | qs to 100 | qs to 100 | qs to 100 | qs to 100 | qs to 100 | qs to 100 |

*Equivalent to 0.3% ciprofloxacin

TABLE 1B

| | EXAMPLE | | | | |
|---|---|---|---|---|---|
| INGREDIENT | 7 | 8 | 9 | 10 | 11 |
| Ciprofloxacin | 0.35* | 0.35* | 0.35* | 0.35* | 0.35* |
| Hydrochloride, | | | | | |
| monohydreate | | | | | |
| Hydrocortisone, | 1.0 | 1.0 | 1.0 | 1.0 | 1.0 |
| micronized | | | | | |
| Benzyl alcohol | 0.9 | 0.9 | 0.9 | 0.9 | 0.9 |
| Polyvinyl Alcohol (AIRVOL | — | 0.05 | 0.1 | 0.5 | 1.0 |
| 205S) 88% hydrolyzed | | | | | |
| Sodium Chloride | 0.9 | 0.9 | 0.9 | 0.9 | 0.9 |
| Sodium Acetate, Trihydrate | 0.68 | 0.68 | 0.68 | 0.68 | 0.68 |
| Glacial Acetic acid | 0.255 | 0.255 | 0.255 | 0.255 | 0.255 |
| Lecithin (Phospholipon | 0.15 | 0.15 | 0.15 | 0.15 | 0.15 |
| 90H) | | | | | |
| Polysorbate 20 | 0.10 | 0.10 | 0.10 | 0.10 | 0.10 |
| Sodium Hydroxide and/or | pH adjust | pH adjust | pH adjust | pH adjust | pH adjust |
| Hydrochloric acid | to 4.7 | to 4.7 | to 4.7 | to 4.7 | to 4.7 |
| Purified Water | qs to 100 | qs to 100 | qs to 100 | qs to 100 | qs to 100 |

*Equivalent to 0.3% ciprofloxacin

The physical stability of suspension formulations is commonly measured in two ways: rate of settling and resuspendability. Rate of settling is assessed by observing the height (in millimeters) of the column of sedimentation visible in a sample contained in a cylinder after shaking and then standing for a period of time. Larger sedimentation heights indicate less separation with less supernatant liquid and less compaction of the insoluble particles. Resuspendability is assessed by measuring the number of inversions (also called strokes) required to redisperse sedimentation which forms after a sample stands undisturbed for a period of time. Rate of settling results for formulations 1-11 are shown in Tables 2 A1 and 2 B1 using the following codes (in order of increasing turbidity): C: Clear Supernatant Phase, LM: Light Milky Phase (less dense than Homogeneous Phase), H: Homogeneous Phase (initial homogeneous phase), D: Dense Phase (more dense than Homogeneous Phase), and S: Sediment: Resuspendability results for the same formulations are shown in Tables 2 A2 and 2 B2.

TABLE 2A1

| Time | Formulation 1 | | Formulation 2 | | Formulation 3 | | Formulation 4 | | Formulation 5 | | Formulation 6 | |
|---|---|---|---|---|---|---|---|---|---|---|---|---|
| Initial | 0-10 mL: | H | 0-10 mL: | H | 0-10 mL: | H | 0-10 mL: | H | 0-10 mL: | H | 0-10 mL: | H |
| 5 min | 0-4 mL: | D | 0-4 mL: | D | 0-10 mL: | H | 0-10 mL: | H | 0-10 mL: | H | 0-10 mL: | H |
| | 4-10 mL: | H | 4-10 mL: | H | | | | | | | | |
| 15 min | 0-1.6 mL: | S | 0-1.9 mL: | S | 0-8.8 mL: | D | 0-2.5 mL: | S | 0-9 mL: | D | 0-10 mL: | H |
| | 1.6-10 mL: | LM | 1.9-10 mL: | LM | 8.8-10 mL: | LM | 2.5-10 mL: | LM | 9-10 mL: | LM | | |
| 30 min | 0-1.5 mL: | S | 0-1.8 mL: | S | 0-7.5 mL: | D | 0-2.4 mL: | S | 0-3.3 mL: | S | 0-9 mL: | D |
| | 1.5-10 mL: | LM | 1.8-10 mL; | LM | 7.5-10 mL: | LM | 2.4-10 mL; | LM | 3.3-10 mL: | LM | 9-10 mL: | LM |
| 45 min | 0-1.5 mL: | S | 0-1.8 mL: | S | 0-7.5 mL: | D | 0-2 mL: | S | 0-2.5 mL: | S | 0-8.5 mL: | D |
| | 1.5-9.5 mL: | LM | 1.8-9.3 mL: | LM | 7.5-10 mL: | LM | 2-9.8 mL: | LM | 2.5-10 mL: | LM | 8.5-10 mL: | LM |
| | 9.5-10 mL: | C | 9.3-10 mL: | C | | | 9.8-10 mL; | C | | | | |
| 1 hour | 0-1.8 mL: | S | 0-1.8 mL: | S | 0-3 mL: | S | 0-1.9 mL: | S | 0-2.4 mL: | S | 0-4 mL: | S |
| | 1.8-8.8 mL: | LM | 1.8-8.8 mL: | LM | 3-10 mL: | LM | 1.9-10 mL: | LM | 2.4-10 mL: | LM | 4-10 mL: | LM |
| | 8.8-10 mL: | C | 8.8-10 mL: | C | | | | | | | | |
| 2 hours | 0-1.8: | S | 0-1.8 mL: | S | 0-2.5 mL: | S | 0-2.5 mL: | S | 0-2.5 mL: | S | 0-3 mL: | S |
| | 1.8-6 mL: | LM | 1.8-7 mL: | LM | 2.5-8.8 mL: | LM | 2.5-10 mL: | LM | 2.5-lOmL: | LM | 3-10 mL: | LM |
| | 6-10 mL: | C | 7-10 m: | C | 8.8-10 mL: | C | | | | | | |
| 3 hours | 0-2.8 mL: | S | 0-5.5 mL: | S | 0-2.2 mL; | S | 0-2 mL; | S | 0-2 mL: | S | 0-2.8: | S |
| | 2.8-10 mL: | C | 5.5-10 mL: | C | 2.2-7.9 mL; | D | 2-10 mL: | C | 2-10 mL: | C | 2.8-10 mL: | C |
| | | | 7.9-10 mL: | C | | | | | | | | |

TABLE 2B1

| Time | Formulation 1 | | Formulation 7 | | Formulation 8 | | Formulation 9 | | Formulation 10 | | Formulation 11 | |
|---|---|---|---|---|---|---|---|---|---|---|---|---|
| Initial | 0-10 mL: | H | 0-10 mL: | H | 0-10 mL: | H | 0-10 mL: | H | 0-10 mL: | H | 0-10 mL: | H |
| 5 min | 0-4 mL: | D | 0-10 mL: | H | 0-10 mL: | H | 0-10 mL: | H | 0-10 mL: | H | 0-10 mL: H | |
| | 4-10 mL; | LM | | | | | | | | | | |
| 10 min | 0-1.9 mL: | S | 0-10 mL: | H | 0-10 mL: | H | 0-10 mL: | H | 0-10 mL: | H | 0-10 mL: | H |
| | 1.9-10 mL: | LM | | | | | | | | | | |
| 15 min | 0-1.7 mL: | S | 0-10 mL: | H | 0-10 mL: | H | 0-10 mL: | H | 0-10 mL: | H | 0-10 mL: | H |
| | 1.7-10 mL: | LM | | | | | | | | | | |
| 20 min | 0-1.7 mL: | S | 0-10 mL: | H | 0-10 mL: | H | 0-10 mL: | H | 0-10 mL: | H | 0-10 mL: | H |
| | 1,7-10 mL: | LM | | | | | | | | | | |
| 30 min | 0-1.5 mL: | S | 0-10 mL: | H | 0-10 mL: | H | 0-10 mL: | H | 0-10 mL: | H | 0-10 mL: | H |
| | 1.5-10 mL: | LM | | | | | | | | | | |
| 45 min | 0-1.5 mL: | S | 0-10 mL: | H | 0-10 mL: | H | 0-10 mL: | H | 0-10 mL: | H | 0-10 mL: | H |
| | 1.5-10 mL: | LM | | | | | | | | | | |
| 1 hour | 0-1.5 mL: | S | 0-10 mL: | H | 0-10 mL: | H | 0-10 mL: | H | 0-10 mL: | H | 0-10 mL: | H |
| | 1.5-10 mL: | LM | | | | | | | | | | |
| 2 hours | 0-1.6 mL: | S | 0-10 mL: | H | 0-10 mL: | H | 0-10 mL: | H | 0-10 mL: | H | 0-10 mL: | H |
| | 1.6-7.5 mL: | LM | | | | | | | | | | |
| | 7.5-10 mL: | C | | | | | | | | | | |
| 3 hours | 0-1.5 mL: | S | 0-9.8 mL: | H | 0-0.5 mL: | S | 0-1 mL: | S | 0-1 mL: | S | 0-0.5 mL: | S |
| | 1.5-5.5 mL: | LM | 9.8-10 mL: | D | 0.5-10 mL: | H | 1-10 mL: | H | 0-2 1-10 mL: | H | 0.5-10 mL: | H |
| | 5.5-10 mL: | C | | | | | | | | | | |
| 4 hours | 0-1.7 mL: | S | 0‥9.8 mL: | H | 0-0.5 mL: | S | 0-1 mL: | S | 0-1 mL: | S | 0-0.5 mL: | S |
| | 1.7-4.5 mL: | LM | 9.8-10 mL: | D | 0.5-10 mL: | H | 1-10 mL: | H | 1-10 mL: | H | 0.5-10 mL: | H |
| | 4.5-10 mL: | C | | | | | | | | | | |
| 5 hours | 0-1.3 mL: | S | 0-9.8 mL: | H | 0-0.5 mL: | S | 0-1 mL: | S; | 0-1 mL: | S | 0-0.5 mL: | S |
| | 1.3-2.5 mL: | LM | 9.8-10 mL: | D | 0.5-10 mL: | H | 1-10 mL: | H | 1-10 mL: | H | 0.5-10 mL: | H |
| | 2.5-10 mL: | C | | | | | | | | | | |
| 6 hours | 0-2 mL: | S | 0-1.5 mL: | S | 0-0.5 mL: | S | 0-0.5 mL | S; | 0-0.5 mL: | S | 0-0.5 mL: | S |
| | 2-10 mL: | C | 1.5-10 mL: | LM | 0.5-10 mL: | LM | 0.5-10 mL | LM | 0.5-10 mL: | LM | 0.5-10 mL: | LM |
| 1 day | 0-1.8 mL: | 5 | 0-1 mL: | 5 | 0-0.5 mL: | S | 0-0.5 mL | S; | 0-0.5 mL: | S | 0-0.5 mL: | S |
| | 1.8-10 mL: | C | 1-9.7 mL: | LM | 0.5-9.7 mL: | LM | 0.5-9.7 mL: | LM | 0.5-9.7 mL: | LM | 0.5-9.7 mL: | LM |
| | | | 9.7-10 mL: | C | 9.7-10 mL: | C | 9.7-10 mL: | C | 9.7-10 mL: | C | 9.7-10 mL: | C |
| 4 day | 0-1.6 mL: | S | 0-0.6 mL: | S | 0-0.5 mL: | S | 0-0.5 mL: | S | 0-0.4 mL: | S | 0-0.3 mL: S | |
| | 1.6-10 mL: | C | 0.6-9.6 mL: | LM | 0.5-9.5 mL: | LM | 0.5-9.8 mL: | LM | 0.4-9.7 mL: | LM | 0.3-9.8 mL: | LM |
| | | | 9.6-10 mL: | C | 9.5-10 mL: | C | 9.8-10 mL: | C | 9.7-10 mL: | C | 9.8-10 mL: | C |

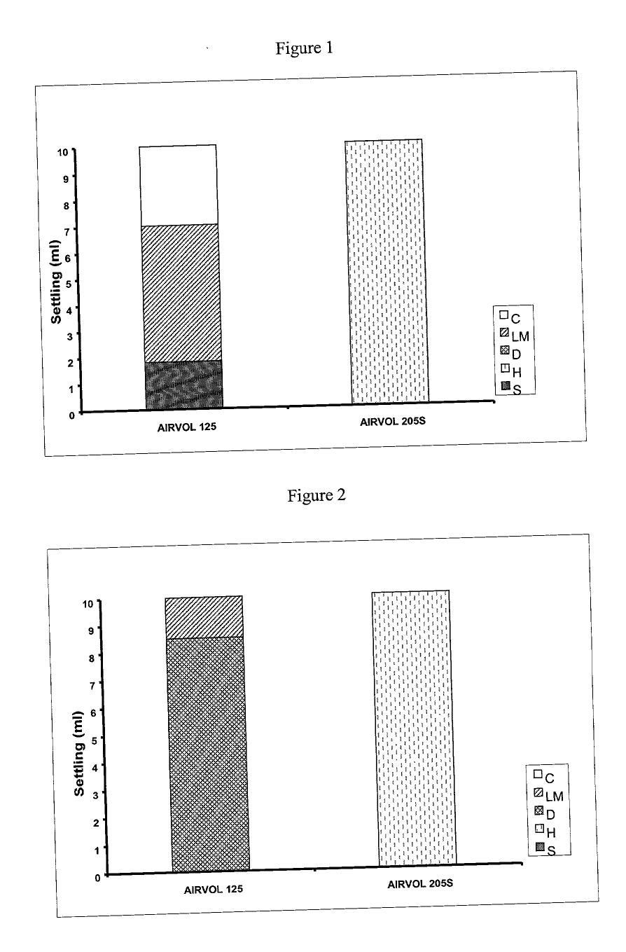

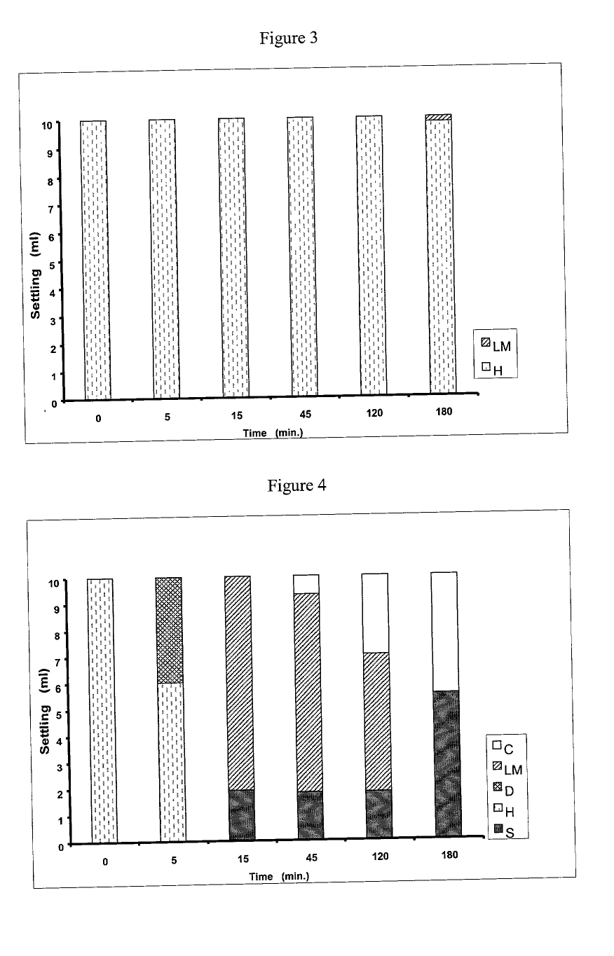

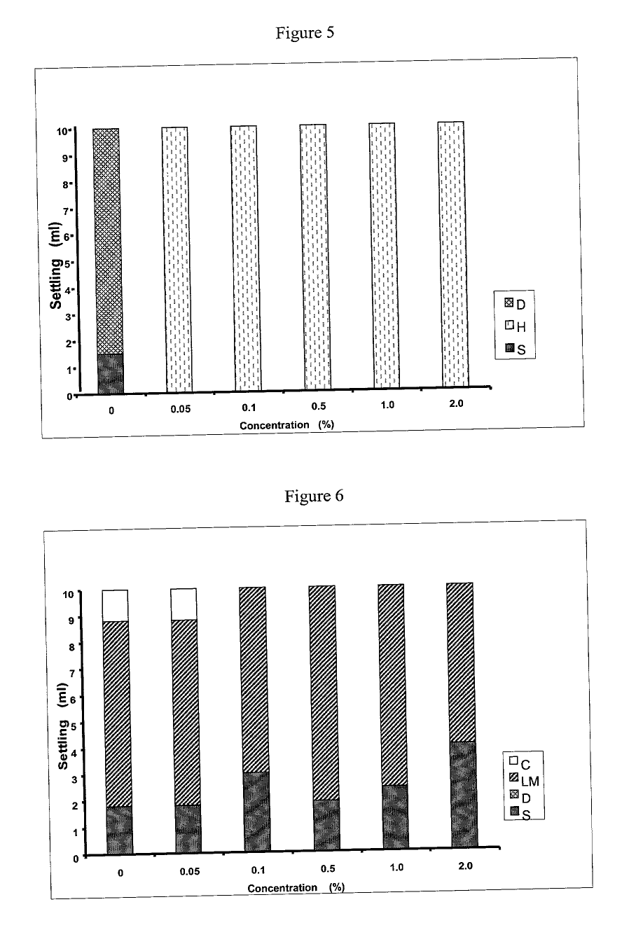

The rate of settling results are summarized in FIGS. 1-6. FIGS. 1 and 2 each compares two different grades of PVA after standing for a specified time. FIG. 1 compares these grades at a concentration 0.05% after standing for 2 hrs. FIG. 2 compares these grades at a concentration of 2% after standing for 45 minutes. FIGS. 3 and 4 compare two grades of PVA at a fixed concentration (0.05%), with varying time (0-180 minutes). FIGS. 5 and 6 compare two grades of PVA at a fixed time (1 hr), with varying concentration (0-2%).

Resuspendability results for Formulations 1-11 are shown in Tables 2A2 and 2B2. Each of the eleven formulations were centrifuged at 500 rpm for 30 minutes using IEC centra-7 centrifuge, then resuspended by gentle, manual inversion technique into a homogenous suspension. The number of manual inversions required to fully resuspend each formulation is presented in Tables 2 A2 and 2 B2.

TABLE 2 A2

| Formulation# | 1 | 2 | 3 | 4 | 5 | 6 |
|---|---|---|---|---|---|---|
| #of | 8,6,6 | 8,7,6 | 5,7,7 | 8,9,11 | 12,12,14 | 37,36,38 |
| Inversions | | | | | | |

TABLE 2 B2

| Formulation# | 7 | 8 | 9 | 10 | 11 |
|---|---|---|---|---|---|
| #of | 9,4,5 | 19,24,25 | 32,28,35 | 35,38,38 | 35,48,46 |
| Inversions | | | | | |

### Example 12

In order to confirm that there was no significant difference in the rate of settling results attributable to the difference in Methods A and B, Formulation #6 was prepared by both methods. The rate of settling results (using the same codes used for Tables 2A1 and 2B1) are shown in Table 3 below.

TABLE 3

| Time | Method A | | Method B | |
|---|---|---|---|---|
| Initial | 0-10 mL: | H | 0-10 mL: | H |
| 5 minutes | 0-10 mL: | H | — | |
| 10 minutes | 0-10 mL: | H | 0-10 mL: | H |
| 15 minutes | 0-10 mL: | H | — | |
| 20 minutes | 0-10 mL: | H | 0-10 mL: | H |
| 30 minutes | 0-9 mL: | D | 0-9 mL: | D |
| | 9-10 mL: | LM | 9-10 mL: | LM |
| 45 minutes | 0-8.5 mL: | D | — | |
| | 8.5-10 mL: | LM | | |
| 1 hour | 0-4 mL: | S | 0-3 mL: | S |
| | 4-10 mL: | LM | 3-10 mL: | LM |
| 2 hours | 0-3 mL: | S | 0-4 mL: | S |
| | 3-10 mL: | LM | 4-10 mL: | LM |
| 3 hours | 0-2.8: | S | — | |
| | 2.8-10 mL: | C | | |

(−) Not Observed

### Example 13

Two grades of polyvinyl alcohol (88% vs. 99% hydrolyzed) were evaluated for the amount of residue (film thickness) they leave upon drying. For topical otic products, the smaller amount of residue (the thinner the film) left upon drying, the less likely a patient is to experience discomfort during use. Separate 2% solutions of AIRVOL 125 (99% hydrolyzed) and AIRVOL 205S (88% hydrolyzed) polyvinyl alcohol were analyzed as follows. The results are shown in Table 4.

Drop Preparation

Using a 10-100 (Eppindorf automatic pipette, 4-5 100 μl drops of each sample were placed into 100×20 mm culture dishes and allowed to dry overnight on the benchtop.

Cross Section Analysis

1. One of the dried drops from the culture dish was removed using a scalpel to separate it from the bottom of the dish.

2. Along its diameter, the drop was cut in half using a very thin razor blade to expose a cross-section.

3. One of the resulting sections was placed between two microscope slides with the cross-section of the drop exposed at the top of the slides.

4. The two microscope slides were bound together using rubber bands.

5. Using modeling clay, the slides were placed in a position under the microscope as to view the cross-section of the sample

6. Using OPTIMAS software, measured the thickness of the film at 100× magnification was measured at the edge of the cross-section and at the center of the cross-section.

TABLE 4
Film Thickness Measurements
of Dried Drops of 2% Polyvinyl Alcohol Compositions

| | Thickness (μm) | |
|---|---|---|
| Sample | Edge of Film | Middle of Film |
| 2% AIRVOL 125 | 52.9, 51.2 | 21.8, 24.9 |
| 2% AIRVOL 205S | 34.6, 45.5 | 7.2, 8.7 |

### Example 16

Films of AIRVOL 125 and AIRVOL 205S were added to 0.1-0.2 ml of phosphate buffered saline and the commercially available otic solutions identified below to determine whether the films would dissolve. The results are shown in Table 5, where “DND” means that the film did not dissolve and “RD” means that the film readily dissolved.

TABLE 5

| | AIRVOL | AIRVOL |
|---|---|---|
| SOLUTION | 125 Film | 125 Film |
| Murine ® Ear Wax Removal System | | |
| Carbamide Peroxide 6.5%, alcohol | DND | DND |
| 6.3%, anhydrous glycerin, polysorbate | | |
| 80 in buffered vehicle | | |
| Star-Otic Ear Solution | | |
| Modified Burrow's solution (Aluminum | DND | Slow |
| acetate), Acetic acid, Boric acid in | | |
| propylene glycol vehicle | | |
| Eckerd Ear Wax Removal Liquid | | |
| Peroxide 6.5%, Citric Acid, Glycerin, | DND | DND |
| Polypropylene Glycol, water & other | | |
| ingredients | | |
| Phosphate buffered saline | DND | RD |

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

We claim:

1. In a topical aqueous composition comprising ciprofloxacin, hydrocortisone, a preservative, a non-ionic surfactant selected from the group consisting of polysorbate 20 to 80, lecithin, and polyvinyl alcohol as a viscosity augmenter, the improvement wherein the polyvinyl alcohol consists essentially of 85-90% hydrolyzed polyvinyl alcohol.

2. The composition of claim 1 wherein the polyvinyl alcohol consists essentially of 88% hydrolyzed polyvinyl alcohol.

3. The composition of claim 1 wherein the preservative is benzyl alcohol.

4. The composition claim 1 wherein the composition comprises 0.01 to 2% (w/w) polyvinyl alcohol.

5. The composition of claim 1 wherein the composition has a pH of about 4.7 and comprises 0.2% (w/w) ciprofloxacin, 1% (w/w) hydrocortisone, 0.9% (w/w) benzyl alcohol, 0.05-1% (w/w) polyvinyl alcohol, a tonicity adjusting agent in the amounts sufficient to cause the composition to have an osmolality of 200-600 mOsm, an acetate buffer, 0.15% (w/w) lecithin and 0.1% (w/w) polysorbate 20.
